(19) United States
(12) Patent Application Publication
(10) Pub. No.: US 2018/0146840 A1
(43) Pub. Date: May 31, 2018

(54) ENDOSCOPIC MEASURING DEVICE

(71) Applicant: New York University, New York, NY (US)

(72) Inventor: Mark POCHAPIN, New York, NY (US)

(21) Appl. No.: 15/799,766

(22) Filed: Oct. 31, 2017

Related U.S. Application Data
(60) Provisional application No. 62/427,522, filed on Nov. 29, 2016.

(51) Int. Cl. A61B 1/005; A61B 1/31; A61B 1/01

(52) U.S. Cl. CPC A61B 1/0051; A61B 17/320016; A61B 1/01; A61B 1/31

(57) ABSTRACT

A device and system for measuring anatomical features includes a flexible member sized to pass slidably through a first lumen of an insertion instrument, a control element, and a measuring device slidably received in the flexible member. The measuring device includes first and second arms moveable between a closed configuration and an open configuration and a measuring element having a first end coupled to the first arm and a second end coupled to the second arm. When the first and second arms are in the closed configuration, the first and second arms are drawn together into a low profile insertion/retraction configuration and, in the open configuration, the measuring filament is pulled taut to a known length selected to measure an anatomical feature. The measuring device is coupled to the distal end of the control element so that forces applied to the control element move the measuring device distally and proximally relative to the flexible member to move the first and second arms between the closed and open configurations.

### PRIORITY CLAIM

This application claims priority to U.S. Provisional Application Serial No. 62/427,522 filed Nov. 29, 2016, the entire contends of which is hereby incorporated by reference herein.

### BACKGROUND

Currently, colonoscopy is the most comprehensive method for colorectal cancer screening, allowing for the detection of potentially pre-cancerous adematous polyps. Colonoscopy has been shown to prevent colorectal cancer and detect cancer early, as evidenced by dramatically declining incidence and mortality rates in recent years. Polyps greater than 10 mm are considered advanced adenomas and have a shorter interval for surveillance endoscopy. Determining the size of polyps and lesions is important for interval assessment and growth. However, conventional screening and treatment methods are often insufficient to reliably measure polyp sizes.

### SUMMARY

The present disclosure relates to an endoscopic measuring device comprising a flexible member sized to pass slidably through a first lumen of an insertion instrument, the flexible member defining a second lumen therein, the flexible member having a flexibility selected to permit insertion of the device to a target site within a living body via a natural body lumen. The endoscopic measuring device also includes a control element extending through the second lumen from a proximal end which remains outside the living body accessible to a user to a distal end. The endoscopic measuring device also includes a measuring device slidably received in the flexible member, the measuring device including first and second arms moveable between a closed configuration and an open configuration and a measuring element having a first end coupled to the first arm and a second end coupled to the second arm, wherein when the first and second arms are in the closed configuration, the first and second arms are drawn together into a low profile insertion/retraction configuration and, in the open configuration, the measuring filament is pulled taut to a known length selected to measure an anatomical feature, the measuring device being coupled to the distal end of the control element so that forces applied to the control element move the measuring device distally and proximally relative to the flexible member to move the first and second arms between the closed and open configurations.

The present disclosure also relates to a method for measuring anatomical features within a living body comprising the steps of: inserting into a living body, a flexible member until a distal end of the flexible member is located adjacent a target anatomical feature to be measured; deploying from the distal end of the flexible member a measuring device including a measuring filament; operating a control element coupled to the measuring device to pull the measuring filament taut, the control member extending to a proximal end which, during use, extends out of the living body for manipulation by a user of the device; and positioning the measuring filament adjacent to the target anatomical feature so that a dimension of the target anatomical feature can be compared to a length of the taut measuring filament.

### BRIEF DESCRIPTION

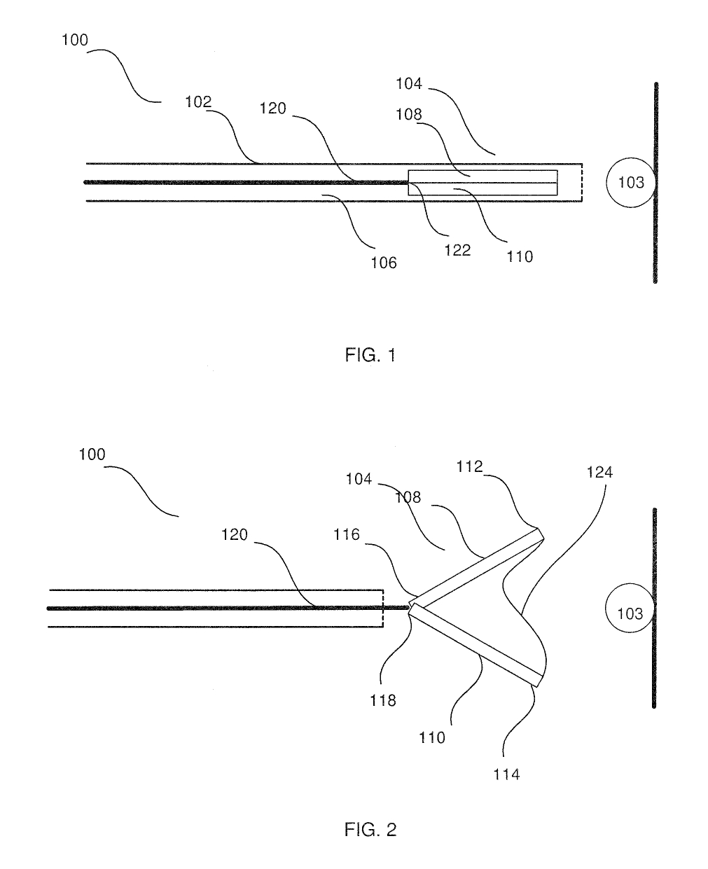

FIG. 1 depicts a partial cross-sectional view of an exemplary endoscopic measuring device according to a first exemplary embodiment of the present disclosure;

FIG. 2 depicts another partial cross-sectional view of the endoscopic measuring device of FIG. 1 in a partially expanded configuration;

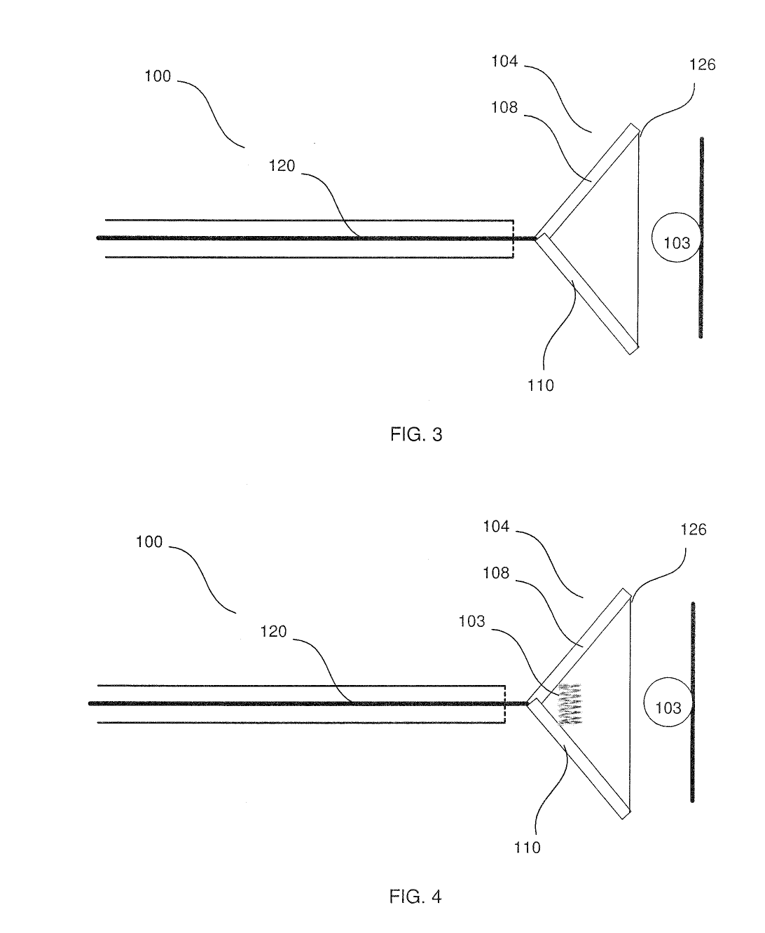

FIG. 3 depicts another partial cross-sectional view of the endoscopic measuring device of FIG. 1 in an expanded configuration;

FIG. 4 depicts a partial cross-sectional view of the endoscopic measuring device of FIG. 1 according to another exemplary embodiment.

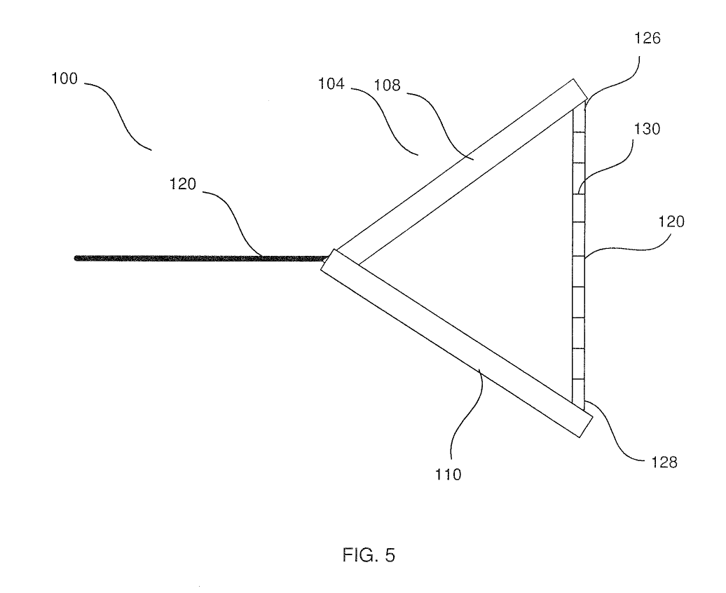

FIG. 5 depicts a side view of an exemplary endoscopic measuring device according to a second exemplary embodiment of the present disclosure;

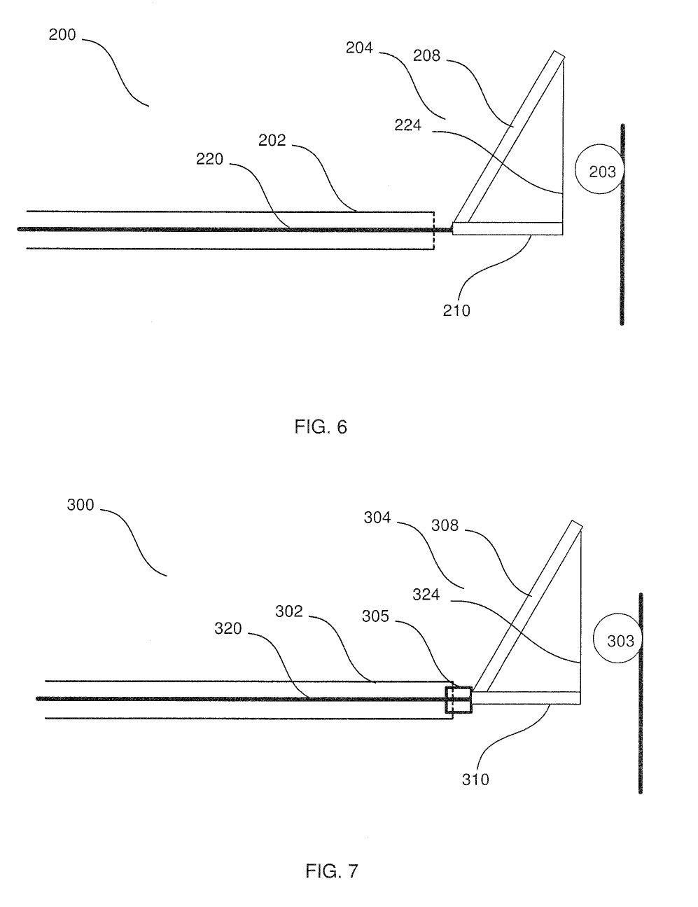

FIG. 6 depicts a partial cross-sectional view of an exemplary endoscopic measuring device according to a third exemplary embodiment of the present disclosure; and

FIG. 7 depicts an exemplary endoscopic measuring device according to a fourth exemplary embodiment of the present disclosure.

### DETAILED DESCRIPTION

The present disclosure may be further understood with reference to the following description and the appended drawings, wherein like elements are referred to with the same reference numerals. The present disclosure is directed to an endoscopic measuring device for measuring anatomic defects such as polyps, perforations and/or lesions. Exemplary embodiments of the present disclosure describe an endoscopic measuring device to allow accurate sizing of lesions in vivo. In particular, exemplary embodiments describe an endoscopic device including arms as movable between a closed configuration and an open configuration with a measuring filament extending between ends of the arms to measure anatomical features during (e.g., polyps encountered during a colonoscopy). The measuring filament may include markings along its length to allow for easier sizing and visualization. It should be noted that the terms “proximal” and “distal,” as used herein, are intended to refer to toward (proximal) and away from (distal) a user of the device.

As shown in FIGS. 1-5, an endoscopic measuring device 100 for measuring anatomic features, such as polyps, comprises a flexible member 102 within which a measuring device 104 is slidably received. The flexible member 102 according to this embodiment is sized, for example, to pass through the working channel of an endoscope for delivery to a target site within the living body. As would be understood by those skilled in the art, the flexible member 102 is preferable sufficiently flexible so that, while the measuring device 104 is received therein, it may be passed along a tortuous path through, for example, a natural body lumen without undue trauma to tissue along and adjacent to the lumen or damage to the measuring device 104 (e.g., through the working channel of a flexible endoscope that has been inserted into the body via a naturally occurring bodily orifice). For example, the flexible member 102 may have a flexibility sufficient to permit the measuring device 104 to be slid through a working channel of a device such as a flexible endoscope and to pass through any bending radii that these devices might achieve. The flexible member 102 may be formed as a flexible sheath defining an internal lumen 106 within which the measuring device 104 is slidably received. In an exemplary embodiment, the flexible member 102 may be formed as a sheath of polyether ether ketone (PEEK) having an outer diameter of 2.2 mm. However, as would be understood by those skilled in the art, other materials and sizes may be used. The flexible member 102 extends between a distal end, within which the measuring device 104 is received, and a proximal end. The proximal end may including a handle (not shown) that, during use, remains outside the body accessible to a user. As would be understood by those skilled in the art, the handle, in an exemplary embodiment, may be configured with a deployment mechanism such as a trigger grip, or any other manner suitable to deploy the measuring device 104 from the internal lumen 106 of the flexible member 102. The measuring device 104 may be actuable by, and deployed from, the flexible member 102 in any suitable member, as described in greater detail below.

Referring to FIG. 1, a measuring device 104 is shown in a low-profile, closed, insertion/withdrawal configuration within the flexible member 102. In an exemplary embodiment, during insertion into the body, the measuring device 104 is held within the flexible member 102 in the insertion/withdrawal configuration. The flexible member 102 may retain the measuring device 104 in any suitable manner, such as by friction fit.

The measuring device 104, according to an exemplary embodiment shown in FIGS. 1-5, is configured as a pair of pivotally connected jaws insertable into a living body through the flexible member 102 to a target tissue to be treated. The measuring device 104 includes first and second arms 108, 110 slidably received within the longitudinal lumen 106 of the flexible member 102. The first and second arms 108, 110 can be moved between a closed configuration, in which the distal ends 112, 114 of the arms 108, 110 are drawn toward one another into a low profile insertion/withdrawal configuration, and an open configuration, shown in FIG. 3, in which the distal ends 112, 114 of the arms 108, 110 are separated from one another by a known distance, as will be further described below, for measuring an anatomical feature 103. The measuring device 104 is coupled to a proximal portion of the device 100, which may include a handle (not shown) that remains outside the body accessible to the user as the measuring device 104 is deployed within the body. The first and second arms 108, 110 are movable between the closed an open configurations via a control member 120 extending into the flexible member 102. The control member 120 extends from a proximal end (not shown), which may be connected to an actuator on the handle, to a distal end 122 coupled to proximal ends 116 of the first and second arms 108, 110.

As further shown in FIG. 1, measuring device 104 includes a measuring filament 124 extending from a first end 126 coupled to a distal end 112 of the first arm 108 to a second end 128 coupled to a distal end 114 of the second arm 110. The measuring filament 124 is a flexible member which may be an elongated cord, ribbon or tape of a predetermined length. Thus, when the measuring filament 124 is drawn taut (when the first and second arms 108, 110, respectively, are moved to the open configuration) the known length of the measuring filament 124 may be compared to adjacent anatomical features to determine the size of the anatomical features. As would be understood, the filament 124 may be flat, cylindrical, or any other appropriate configuration. In an exemplary embodiment, the filament 124 is formed of a flexible material such as, for example polyethelene, so that when the first and second arms 108, 110 are in the closed position, the filament 124 becomes slack and may be folded into the flexible member 102, as shown in FIG. 2. In an exemplary embodiment, the measuring filament 124 is between 0.75 and 1.25 cm long, and more specifically, is 1 cm long. However, it is understood that the measuring filament 124 may be any desired length, depending on the size of the anatomical feature 103 to be measured. To facilitate easy and accurate measurements, the measuring filament 124 may include markings 130, such as slashes, along its length, as shown in FIG. 5. In an embodiment, the markings 130 may occur at 1 mm intervals. In another embodiment, smaller markings (not shown) may occur at 0.1 mm intervals between each 1 mm markings 130. However, it is understood that the markings 130 may take any form and occur at any desired interval. The measuring filament 124 is coupled to distal ends 112, 114 of arms 108, 110 so that when arms 108, 110 are in the open configuration, filament 124 is pulled taut to its selected length to measure a target anatomical feature 103 placed next to the filament 124, as shown in FIG. 3. Because the filament 124 is coupled to the distal ends 112, 114 of the first and second arms 108, 110, respectively, the filament 124 acts as a limiting member so that first and second arms 108, 110 may be opened only to the extent permitted by the filament 124. The filament 124 may be coupled to the first and second arms 108, 110 in any known manner such as, for example, by adhesive or welding. As would be understood by those skilled in the art, if the filament 124 includes markings 130, the markings 130 are positioned along (which surface?) of the filament 124 so as to be easily viewable by the user (e.g., on a side of the filament 124 in the same plane as the first and second arms 108, 110.

According to an exemplary method, the flexible member 102, with the measuring device 104 held therein, is inserted to a target area within a living body, (e.g., through a working channel of a flexible endoscope). The measuring device 104 is maintained in the insertion/withdrawal configuration during insertion to facilitate its passage through the insertion instrument (e.g., endoscope). Once the flexible member 102 has reached the target area, the measuring device 103 is moved distally out of a distal end of the flexible member 102 by advancing the control wire 120 distally and the first and second arms 108, 110 are moved to the open configuration by moving the control member 120 distally relative to the flexible member 102. In an exemplary embodiment shown in FIG. 4, the measuring device 104 may include a spring or other flexible biasing member 103 disposed between the first and second arms 108, 110 to bias the arms 108, 110 toward and open configuration and keep the arms 108, 110 taut. Alternatively, the first and second arms 108, 110 may be formed of a single piece of material biased toward the open configuration so that, when the measuring device 104 is advanced distally out of the flexible member 102, the first and second arms 108, 110 automatically move to the open configuration. The arms 108, 110 may then be moved back to the insertion/withdrawal configuration by simply drawing the measuring device 104 proximally back into the flexible member 102. The control member 120, which extends proximally out of the living body, may be manipulated by the user via, for example, the handle.

Once the measuring filament 124 has been pulled taut to its known length, the filament 124 is positioned adjacent to the target anatomical feature 103 so that a dimension of the target anatomical feature 103 may be compared to a length of the measuring filament 124. In an exemplary embodiment, markings 130 along the length of measuring filament 124 are viewable by the user to accurately measure the anatomical feature 103 to a more exact degree. Once measurement of the anatomical feature 103 has been taken, the arms 108, 110 are moved toward the closed configuration by drawing the control member 120 proximally to retract the measuring device 104 proximally into the flexible member 102. The flexible member 102 may then be withdrawn from the body.

According to an alternate embodiment, as shown in FIG. 6, a device 200 is substantially similar to the device 100 described above, comprising a measuring device 204 including arms 208, 210 slidably received within a flexible member 202 to be moved between an open configuration and a closed configuration via a control member 220. Rather than having two arms of equal length however, arm 208 has a length that is greater than the length of arm 210, as can be seen in FIG. 6. Arm 208 may have a length of, for example, 10 mm, while arm 210 has a length of, for example, 5 mm. Arm 208 extends laterally away from the longitudinal axis of the flexible member a greater distance than arm 210 so that measuring filament 224 is off-center with respect to the flexible member 202. In an embodiment, arm 210 may extend along the longitudinal axis of the flexible member 202 while arm 208 extends laterally outward at an angle of, for example, 45 to 90 degrees, and more specifically, an angle of 60 degrees. In another exemplary embodiment, arm 210 may extend laterally from the longitudinal axis of the flexible member 202 at an angle of, for example, 30 degrees, while arm 208 extends laterally in an opposing direction from the longitudinal axis of the flexible member 202 at an angle of, for example, 60 degrees. As with measuring device 104, in an exemplary embodiment the length of measuring filament 224 is 10 mm such that arms 208, 210 expand to this selected length. However, it is understood that filament 224 may have any selected length in accordance to the anatomical feature 203 to be measured.

According to an alternate embodiment, as shown in FIG. 7, a device 300 is substantially similar to the devices 100, 200 described above, comprising a measuring device 304 including arms 308, 310 slidably received within a flexible member 302 to be moved between an open configuration and a closed configuration via a control member 320. In this embodiment, the device 300 may include a rotation mechanism (305) coupled to the measuring device 304 such that the measuring device 304 may be rotated to more easily measure an anatomical feature 303 in any necessary direction. In an exemplary embodiment, measuring device 304 may be rotated by rotation of the handle (not shown), which is non-rotatably coupled to the control member 320. Rotation of the control member 320, which is non-rotatably coupled to the measuring device 304, in turn rotates the measuring device 304.

The devices 200, 300 may be used in a manner substantially similar to the device 100. In particular, the arms 208, 210, 308, 310 may be used to measure a target anatomical feature as described above.

Variations may be made in the structure and methodology of the present disclosure, without departing from the spirit and the scope of the disclosure. Thus, it is intended that the present disclosure cover the modifications and variations of this disclosure that may be contemplated by a person of skill in the art.

What is claimed is:

1. An endoscopic measuring device, comprising:
a flexible member sized to pass slidably through a first lumen of an insertion instrument, the flexible member defining a second lumen therein, the flexible member having a flexibility selected to permit insertion of the device to a target site within a living body via a natural body lumen;
a control element extending through the second lumen from a proximal end which remains outside the living body accessible to a user to a distal end; and
a measuring device slidably received in the flexible member, the measuring device including first and second arms moveable between a closed configuration and an open configuration and a measuring filament having a first end coupled to the first arm and a second end coupled to the second arm, wherein when the first and second arms are in the closed configuration, the first and second arms are drawn together into a low profile insertion/retraction configuration and, in the open configuration, the measuring filament is pulled taut to a known length selected to measure an anatomical feature, the measuring device being coupled to the distal end of the control element so that forces applied to the control element move the measuring device distally and proximally relative to the flexible member to move the first and second arms between the closed and open configurations.

2. The device according to claim 1, wherein the flexible member is sized and shaped to pass through a working channel of a flexible endoscope.

3. The device according to claim 1, wherein the measuring filament is coupled to distal ends of the first and second arms.

4. The device according to claim 3, wherein the first arm has a length greater than the second arm and wherein, in the open configuration, a distal end of the first arm extends laterally away from a longitudinal axis of the flexible member by a distance greater than distal end of the second arm so that the filament is off-center with respect to the flexible member.

5. The device according to claim 1, wherein the measuring device has a length of 10 mm when in the open configuration.

6. The device according to claim 1, wherein the measuring filament includes a plurality of first markings extending from the first end to the second end at 1 mm intervals.

7. The device according to claim 6, wherein the measuring filament further includes a plurality of second markings extending from the first end to the second end at 0.1 mm intervals.

8. The device according to claim 1, further comprising a rotation mechanism coupled to the measuring device for rotation thereof.

9. The device according to claim 1, further comprising a handle coupled to the control member.

10. The device according to claim 9, wherein the handle is non-rotatably coupled to the rotation mechanism such that rotation of the handle rotates the measuring device.

11. A method for measuring anatomical feature within a living body, comprising the steps of:
inserting into a living body, a flexible member until a distal end of the flexible member is located adjacent a target anatomical feature to be measured;
deploying from the distal end of the flexible member a measuring device including a measuring filament; and
operating a control element coupled to the measuring device to pull the measuring filament taut, the control member extending to a proximal end which, during use, extends out of the living body for manipulation by a user of the device; and
positioning the measuring filament adjacent to the target anatomical feature so that a dimension of the target anatomical feature can be compared to a length of the taut measuring filament.

12. The method according to claim 11, wherein the flexible member is inserted into the body through an insertion instrument extending through a natural body lumen accessed via a natural bodily orifice.

13. The method according to claim 11, wherein the measuring device includes first and second arms moveable between a closed configuration and an open configuration, the measuring filament being coupled to distal ends of the first and second arms, further comprising moving the first and second arms from a closed configuration in which the distal ends of the first and second arms are drawn together into a low profile insertion/retraction position to an open configuration in which the distal ends of the first and second arms are spread apart from one another to pull the measuring filament taut to a known length selected to measure an anatomical feature.

14. The method according to claim 13, wherein the measuring filament is coupled to distal ends of the first and second arms.

15. The method according to claim 13, wherein the first arm has a length greater than the second arm and wherein, in the open configuration, a distal end of the first arm extends laterally away from a longitudinal axis of the flexible member by a distance greater than distal end of the second arm so that the filament is off-center with respect to the flexible member.

16. The method according to claim 13, wherein the measuring device has a length of 10 mm when in the open configuration.

17. The method according to claim 11, wherein the measuring filament includes a plurality of first markings extending from the first end to the second end at 1 mm intervals.

18. The method according to claim 17, wherein the measuring filament further includes a plurality of second markings extending from the first end to the second end at 0.1 mm intervals.

19. The method according to claim 11, wherein a rotation mechanism is coupled to the measuring device for rotation thereof.

20. The method according to claim 11, wherein a handle is coupled to the control member.

21. The method according to claim 20, wherein the handle is non-rotatably coupled to the rotation mechanism such that rotation of the handle rotates the measuring device.
